(19) United States
(12) Patent Application Publication
(10) Pub. No.: US 2013/0108697 A1
(43) Pub. Date: May 2, 2013

(54) MODIFIED RELEASE DOSAGE FORM

(71) Applicants: Ramakant Kashinath Gundo, Ahmednagar (IN); Rahul Sudhakar Dabre, Nagpur (IN); Girish Kumar Jain, Delhi (IN)

(72) Inventors: Ramakant Kashinath Gundo, Ahmednagar (IN); Rahul Sudhakar Dabre, Nagpur (IN); Girish Kumar Jain, Delhi (IN)

(21) Appl. No.: 13/582,449

(22) PCT Filed: Mar. 2, 2011
(86) PCT No.: PCT/IB2011/050880

§ 371 Date: Dec. 21, 2012

(30) Foreign Application Priority Data
Mar. 4, 2010 (IN) 571/MUM/2010
Mar. 4, 2010 (IN) 572/MUM/2010

(51) Int. Cl. A61K 9/00

(52) U.S. Cl. CPC A61K 9/0002
USPC 424/468; 514/242; 514/266.24; 514/252.17; 514/649

(57) ABSTRACT

There is provided a modified release dosage form comprising a core coated with a polymeric coat, said polymeric coat comprising one or more rate controlling polymers, said dosage form having a hole extending through the dosage form resulting in an inner radial surface and an outer radial surface, said core comprising at least one therapeutically active ingredient, characterized in that the inner radial surface is partially coated with said polymeric coat.

### FIELD OF THE INVENTION

There is provided a modified release dosage form comprising a core coated with a polymeric coat, said polymeric coat comprising one or more rate controlling polymers, said dosage form having a hole extending through the dosage form resulting in an inner radial surface and an outer radial surface, said core comprising at least one therapeutically active ingredient, characterized in that the inner radial surface is partially coated with said polymeric coat. The invention also provides the process for preparation of such compositions.

### BACKGROUND OF THE INVENTION

Development of pharmaceutical drug delivery systems capable of modulating release of active ingredients to achieve desired optimum therapeutic effect has been an important area of research. In contrast to immediate release, which dumps the active ingredient for absorption in gastro-intestinal tract, the goal of a modified release dosage form is to deliver the active ingredient at a predetermined rate over a period of time.

Various attempts have been made by several researchers for development of a delivery system modifying the release of active ingredients providing desired therapeutic effect for prolong period of time with reduced frequency of administration and achieve better patient compliance.

Lamotrigine is a drug effective for the treatment of CNS disorders particularly effective in the treatment of epilepsy, pain, psychiatric disorders, etc. Chemically Lamotrigine is 3,5-diamino-6-(2,3-dichlorophenyl)-as-triazine. It is marketed in different tablet dosage forms including form of tablet, chewable tablet, orally disintegrating tablet and extended release tablet dosage form. It is available under brand names such as Lamictal® or Lamictal XR® by Smithkline Beechem.

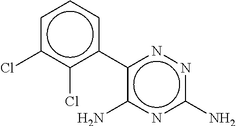

A variety of approaches have been suggested in the art to provide a modified release dosage form such as those disclosed in U.S. Pat. Nos. 4,816,262; 6,110,500 and 6,352,721.

U.S. Pat. Nos. 5,863,558; 5,861,179, US Patent Application Nos. 20040043996, 20090022789, 20090004229, 20070196491, 20080020041, and 20090196924 disclose modified release dosage form for an antiepileptic drug including Lamotrigine. US Application No. 20040192690 disclose a sustained release formulation of Lamotrigine based on Diffcore® technology.

The U.S. Pat. Nos. 5,906,832 and 5,660,861 disclose osmotic dosage forms providing modified release of active ingredients including Lamotrigine.

European Patent No. 01063971B1, U.S. Patent Application Nos. 20020086054, 20040010000, 20020002364 and PCT Publication No. 9961005A1 disclose various osmotic dosage forms providing controlled the release of active ingredients.

U.S. Pat. No. 5,137,731 discloses chemical tablet having upper and lower annular surfaces connected by a side surface at junctures, the upper and lower annular surfaces being partially encased in a plastic covering. The tablet has a central hole to aid in water flow inside.

U.S. Pat. No. 5,376,771 discloses process for producing pharmaceutical drug delivery dosage form having plurality of apertures in its outside surface, the apertures providing an egress for a drug when the drug delivery device is placed in a biological environment of use, the process uses digital laser marking system.

U.S. Patent Application Nos. 20050169994 and 20060182804 disclose a controlled release delivery formulation comprising a core containing Carvedilol, a release modifying agent; and an outer semipermiable coating including orifice extending substantially through the outer coating but not penetrating the core allowing for release of the Carvedilol into the environment of use.

U.S. Patent Application No. 20080003287 and PCT Publication No. 2008005020A1 disclose extended release tablet with perforated core with a hole extending through the core. U.S. Application No. 20050025829A1 discloses perforated controlled release zero order release matrix tablet comprising one or more enteric polymer mixed with active ingredient.

PCT Publication No. 1999048481A1 discloses a donut shaped controlled release tablet with a cylindrical hole extending through the center of core. The core comprises active ingredient and at least one hydrophilic, water-soluble, polymeric carrier; and a coating of hydrophobic, water-insoluble material covering the core except the surface of core surrounding the cylindrical hole.

U.S Patent Application No. 20090004281A1 and PCT Publication No. 2010015840A1 disclose osmotic dosage form.

The prior art modified release dosage forms with a central perforation or hole for releasing therapeutically active ingredients generally release a disproportionate amount of therapeutically active ingredients quickly after ingestion by a patient and fail to provide consistent release profile. The technology used in developing most of such dosage forms involves compression of tablet core followed by creating a perforation or hole in the coated or uncoated core by drilling or other means. These dosage forms provide brisk release of active ingredient through the perforation or hole instead of a consistent zero order release kinetics. Such systems are prone to variation in release kinetics and may result in a quick spike in the level of medication in the patient's bloodstream. However, there are many instances where a spike in the medication level in a patient is undesirable, as where pharmaceuticals are used to treat a chronic condition. Further, extended release of therapeutically active agents is highly desirable for agents that have characteristically short half-lives.

The cost involved in producing the prior art dosage forms often use expensive techniques and precision equipments such as laser drilling, mechanical drilling, ultrasonic technology and the like. Therefore, there exists a need for a dosage form that will provide desired consistent release kinetics with a good bioavailability of therapeutically active pharmaceutical ingredients of various classes along with increased duration of action and decreased frequency of dosing. Also there exists a need of cheaper and more economical platform technology, which can provide the optimized pharmacokinetic profiles of the dosage forms by simple variation in the system dimensions, excipients used and processes involved therein.

The need for modified release dosage forms is particularly of importance in case of drugs such as Lamotrigine, which is rapidly and completely absorbed after oral administration with negligible first pass metabolism. The immediate release formulations of Lamotrigine often produce brisk release of Lamotrigine in the stomach resulting in cyclical plasma concentration and pharmacokinetic profile with peaks occurring after administration followed by troughs occurring before the next administration of drug. In particular for the treatment of epilepsy wherein the Lamotrigine is a drug of choice, it is speculated that the troughs may lead to breakthrough seizures and the peak plasma concentration may result in some adverse events occurring in some patients. Such deviations in the mean plasma concentrations may not be desired.

The present inventors have now developed a modified release pharmaceutical dosage form comprising active ingredient, which helps to control initial burst release of the active ingredient in acidic media and achieves desirable dissolution profile and consistent release pattern. The modified release dosage form of the present invention comprise a core coated with a polymeric coat, said polymeric coat comprising one or more rate controlling polymers, said dosage form having a hole extending through the dosage form resulting in an inner radial surface and an outer radial surface, said core comprising at least one therapeutically active ingredient, characterized in that the inner radial surface is partially coated with said polymeric coat. Modified release pharmaceutical dosage form comprising lamotrigine as an active ingredient was particularly developed which provided better results compared to conventional lamotrigine tablets.

### SUMMARY OF THE INVENTION

In one general aspect of the invention, there is provided a modified release dosage form comprising a core coated with a polymeric coat, said polymeric coat comprising one or more rate controlling polymers, said dosage form having a hole extending through the dosage form resulting in an inner radial surface and an outer radial surface, said core comprising at least one therapeutically active ingredient, characterized in that the inner radial surface is partially coated with said polymeric coat.

In another general aspect of the invention there is provided a modified release dosage form comprising a core coated with a polymeric coat, said polymeric coat comprising one or more rate controlling polymers, said dosage form having a hole extending through the dosage form resulting in an inner radial surface and an outer radial surface, said core comprising at least one therapeutically active ingredient, characterized in that the inner radial surface is partially coated with said polymeric coat, further characterized in that the ratio of outer radial surface area to the inner radial surface area is between about 20 to about 1.

In another general aspect of the invention there is provided a modified release dosage form comprising a core coated with a polymeric coat, said polymeric coat comprising one or more rate controlling polymers, said dosage form having a hole extending through the dosage form resulting in an inner radial surface and an outer radial surface, said core comprising at least one therapeutically active ingredient, characterized in that the inner radial surface is partially coated with said polymeric coat, and further comprising at least one pharmaceutically acceptable excipient.

In another general aspect of the invention, there is provided a modified release dosage form comprising a core coated with a polymeric coat, said polymeric coat comprising one or more rate controlling polymers, said dosage form having a hole extending through the dosage form resulting in an inner radial surface and an outer radial surface, said core comprising lamotrigine as an active ingredient, characterized in that the inner radial surface is partially coated with said polymeric coat.

In another general aspect of the invention there is provided a modified release dosage form comprising a core coated with a polymeric coat, said polymeric coat comprising one or more rate controlling polymers, said dosage form having a hole extending through the dosage form resulting in an inner radial surface and an outer radial surface, said core comprising lamotrigine as an active ingredient, characterized in that the inner radial surface is partially coated with said polymeric coat, further characterized in that the ratio of outer radial surface area to the inner radial surface area is between about 20 to about 1.

In yet another general aspect of the invention there is provided a modified release dosage form comprising a core coated with a polymeric coat, said polymeric coat comprising one or more rate controlling polymers, said dosage form having a hole extending through the dosage form resulting in an inner radial surface and an outer radial surface, said core comprising lamotrigine as an active ingredient, characterized in that the inner radial surface is partially coated with said polymeric coat, and further comprising at least one pharmaceutically acceptable excipient.

One of the embodiments of the modified release dosage form may include one or more of the following features. The modified release dosage forms of the present invention may be present in different dosage forms. The modified release dosage forms of the present invention may be present in different shapes.

### DETAILED DESCRIPTION OF THE INVENTION

The term “modified release” as used herein refers to and includes a prolonged release, extended release, controlled release, controlled delivery, slow release, sustained release and delayed release.

The term “dosage form” as used herein refers to a pharmaceutical preparation in which dose or doses of one or more therapeutically active ingredients are included.

The term “rate controlling polymer” as used herein refers to and includes mean a polymer capable of altering, modulating or modifying rate of release of therapeutically active ingredient from the dosage form. The term “rate controlling polymers” includes pharmaceutically acceptable hydrophilic polymers, hydrophobic polymers, pH independent polymers, pH dependent polymers, soluble polymers, insoluble polymers, lipids or lipidic materials or combinations thereof. The polymer may be a homopolymer or a copolymer. The amount of rate controlling polymer used in this invention depends on various factors including desired release profile. In some embodiment of this invention, the dosage form comprises about 1% to about 25% rate controlling polymer(s) by weight of core.

The term “therapeutically active ingredient” as used herein refers to and includes any active pharmaceutical agent or a pharmaceutically acceptable derivative thereof including salts, isomers, mixtures, hydrates, pro-drugs, esters, polymorphs and so on. In some embodiments suitable pharmaceutically acceptable salts include but are not limited to the chloride and other halogen salts, and salts such as are formed by reaction of therapeutically active ingredient with acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pathothenic, phosphoric, p-toluenesulfonic, succinic, sulfuric, and tartaric acids, and the like. The amount of therapeutically active ingredient in the core largely depends on the extent and duration of the therapeutic response desired and can vary depending on various factors, including characteristics of the subject (for example height, weight, sex, age and medical history of the patient). In some embodiment of this invention the dosage form comprises 0.1 to 90% of therapeutically active ingredient.

The term “Lamotrigine active ingredient” as used herein refers to and includes Lamotrigine or a pharmaceutically acceptable derivative thereof including salts, isomers, racemic mixtures, hydrates, pro-drugs, esters, polymorphs or mixtures thereof. In some embodiments suitable pharmaceutically acceptable salts include but are not limited to the chloride and other halogen salts, and salts such as are formed by reaction of lamotrigine with acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pathothenic, phosphoric, p-toluenesulfonic, succinic, sulfuric, and tartaric acids, and the like. The amount of Lamotrigine active ingredient in the core largely depends on the extent and duration of the therapeutic response desired and can vary depending on various factors, including characteristics of the subject (for example height, weight, sex, age and medical history of the patient). In some embodiment of this invention the dosage form comprises 0.1 to 90% of Lamotrigine active ingredient.

Accordingly, there is provided a modified release dosage form comprising a core coated with a polymeric coat, said polymeric coat comprising one or more rate controlling polymers, said dosage form having a hole extending through the dosage form resulting in an inner radial surface and an outer radial surface, said dosage form comprising at least one therapeutically active ingredient, characterized in that the inner radial surface is partially coated with said polymeric coat.

The dosage form according to this invention comprises a core coated with a polymeric coat. The term “core” as used herein refers to a structure that is surrounded by a wall, membrane, or coating. The wall, membrane, or coating can be a functional or non-functional coating. In some embodiments, the core comprises at least one therapeutically active ingredient. In some other embodiments, the core comprises one or more pharmaceutically active excipients. In some embodiments, the core comprises one or more swellable hydrophilic polymers, water soluble and/or insoluble agents.

Typical non-limiting examples of swellable hydrophilic polymers include cellulose derivatives such as hydroxypropylmethyl cellulose (HPMC), hydroxypropyl cellulose, hydroxyethyl cellulose, methyl cellulose, ethyl cellulose, carboxymethyl cellulose, carboxyethyl cellulose, carboxymethyl hydroxyethyl cellulose, microcrystalline cellulose, polyethylene oxides, polyvinylpyrrolidone, polyalkylene glycols, gelatine, polyvinyl alcohol, starch and derivatives thereof, acrylic acid polymers, polymethacrylates, polysaccharides such as xanthan gum, tragacanth gum, gum karaya, guar gum, acacia, gellan gum locust bean gum, alkali metal salts of alginic acid or pectic acid, sodium alginate, potassium alginate, ammonium alginate, chitosan, maleic anhydride copolymers, poly(ethyleneimine), polyurethane hydrogels, crosslinked polyacrylic acids, derivatives and the like.

Typical, non-limiting examples of water soluble agents include one or more of lactose anhydrous, lactose monohydrate, mannitol, sodium chloride, sucrose and the like. Typical, non-limiting examples of water insoluble agents include stearic acid, calcium stearate, magnesium stearate, zinc stearate, sodium stearyl fumarate and the like.

The core is coated with a polymeric coat, said coat comprising one or more rate controlling polymers. The coat comprising one or more rate-controlling polymer is capable of altering, modifying or modulating rate of release of therapeutically active ingredient from the dosage form. Typical, non-limiting examples of rate controlling polymers include pharmaceutically acceptable hydrophilic polymers, hydrophobic polymers, pH independent polymers, pH dependent polymers, soluble polymers, insoluble polymers, lipids or lipidic materials or combinations. The rate controlling polymer may be a homopolymer or a copolymer or a mixture thereof. Typical, non-limiting examples of rate controlling polymers include polyvinyl acetate, cellulose acetate, cellulose acetate butyrate, cellulose acetate propionate, ethyl cellulose, a fatty acid, a fatty acid ester, an alkyl alcohol, a wax, shellac, rosin, zein (prolamine from corn), povidone, kollidon SR, poly(meth)acrylate, microcrystalline cellulose or poly(ethylene oxide), polyuronic acid salts, cellulose ethers, xanthan gum, tragacanth gum, gum karaya, guar gum, acacia, gellan gum locust bean gum, alkali metal salts of alginic acid or pectic acid, sodium alginate, potassium alginate, ammonium alginate, hydroxypropyl cellulose, hydroxy ethyl cellulose, hydroxypropyl methyl cellulose, carboxyvinyl polymers, polymerized gelatin, shellac, methacrylic acid copolymer, cellulose butyrate phthalate, cellulose hydrogen phthalate, cellulose propionate phthalate, polyvinyl acetate phthalate (PVAP), cellulose acetate phthalate (CAP), cellulose acetate trimellitate (CAT), hydroxypropyl methylcellulose phthalate, hydroxypropyl methylcellulose acetate, dioxypropyl methylcellulose succinate, carboxymethyl ethyl cellulose (CMEC), hydroxypropyl methylcellulose acetate succinate (HPMCAS), and acrylic acid polymers and copolymers like methyl acrylate, ethyl acrylate, methyl methacrylate, ethyl methacrylate, copolymers of acrylic and methacrylic acid esters, methacrylic acid copolymer type C (Eudragit L 100 55), methacrylic acid copolymer type C (Eudragit L 30D 55), methacrylic acid copolymer type A (Eudragit L 100), and methacrylic acid copolymer.

The dosage form according to this invention has a hole extending through the dosage form resulting in an inner radial surface and an outer radial surface. The hole refers to a space, aperture or hollow portion in the dosage form extending through the dosage form and can be of any geometrical shape, including for example, circular, oval, donut, rectangular, triangular, cylindrical, pentagonal, hexagonal, octagonal, trapezoid, combinations thereof and the like. The inner radial surface is the surface surrounding the hole. The outer radial surface refers to the total surface of the dosage form excluding the inner radial surface.

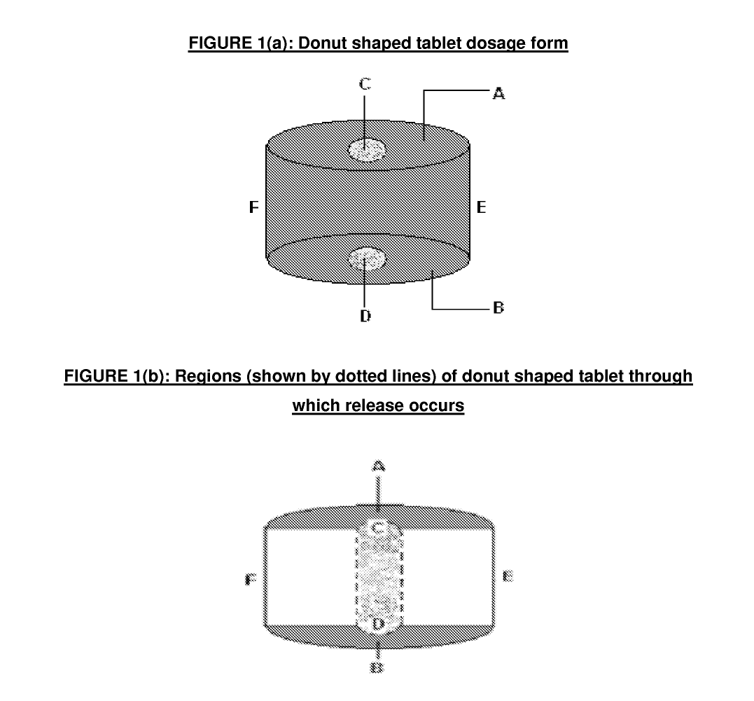

FIG. 1(a) is a general view of a dosage form designed as donut shaped tablet for oral administration of therapeutically active pharmaceutical ingredient. The regions A and B designate outer surface including upper annular surface and lower annular surface of the dosage form. The inner region extending from C to D designates a hole or hollow portion in the dosage form extending through the dosage form. The outer region extending from E to F designates the outer radial surface of dosage form. FIG. 1(b) shows the partially coated region extending from C to D of inner surface of donut shaped tablet through which controlled release of therapeutically active pharmaceutical ingredient occurs.

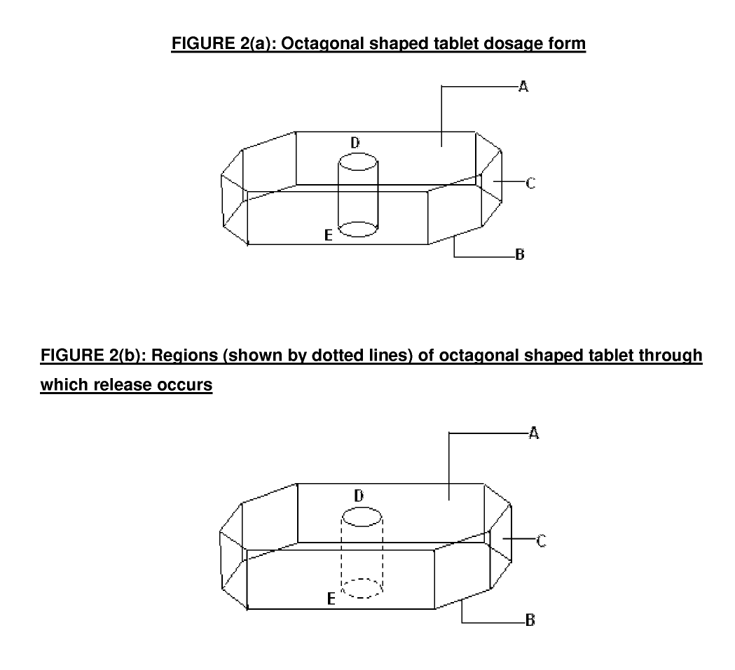

FIG. 2(a) is a general view of a dosage form designed as octagonal shaped tablet for oral administration of therapeutically active pharmaceutical ingredient. The regions A, B and C (all side surfaces) designate the outer surfaces of dosage form. The inner region extending from D to E designates a hole or hollow portion in the dosage form extending through the dosage form. FIG. 2(b) shows the regions (shown by dotted lines) of inner surface of octagonal shaped tablet through which controlled release of therapeutically active pharmaceutical ingredient occurs.

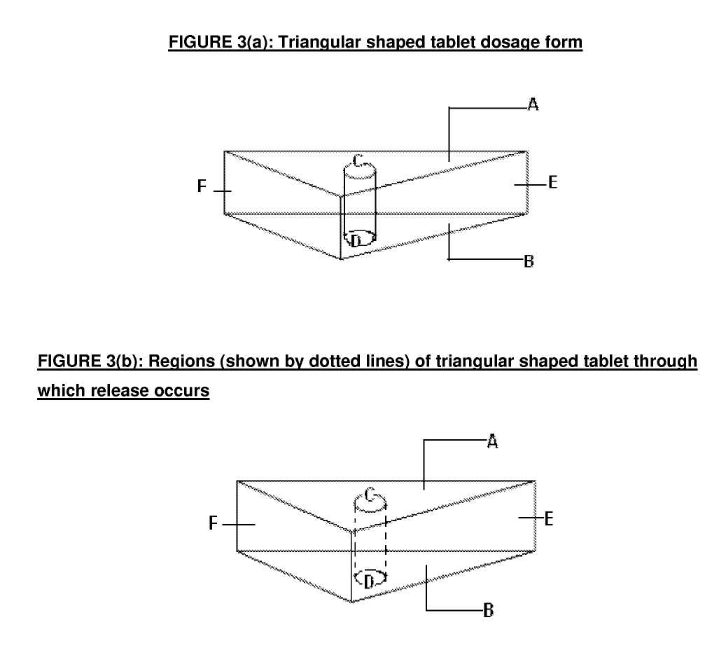

FIG. 3(a) is a general view of a dosage form designed as triangular shaped tablet for oral administration of therapeutically active pharmaceutical ingredient. The regions A, B, E and F designate the outer surfaces of dosage form. The inner region extending from C to D designates a hole or hollow portion in the dosage form extending through the dosage form. FIG. 3(b) shows the regions (shown by dotted lines) of inner surface of triangular shaped tablet through which controlled release of therapeutically active pharmaceutical ingredient occurs.

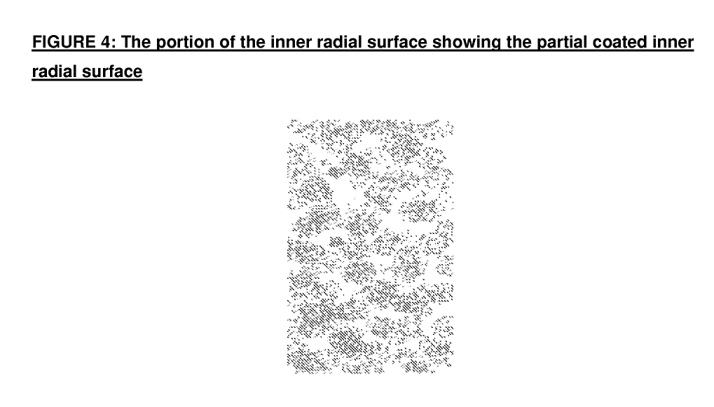

The inner radial surface of the dosage form according to this invention is partially coated with a polymeric coat comprising one or more of rate controlling polymers. The term partially coated refers to the extent of coating on the inner radial surface such that at least some surface of the inner radial surface remains uncoated. See, for example, FIG. 1(b) and FIG. 4. FIG. 4 shows the enlarged portion of the inner radial surface showing the partially coated inner radial surface.

One way to get the dosage form according to this invention with a partially coated inner surface is to vary the ratio of outer radial surface area to the inner radial surface area of the dosage form. In some embodiments, the ratio of outer radial surface area to the inner radial surface area is between about 20 to about 1. In some other embodiments, the ratio of outer radial surface area to the inner radial surface area is about 7.

In some embodiments, the modified release dosage form according to this invention comprises at least one pharmaceutically acceptable excipient. Typical, non-limiting examples of pharmaceutically acceptable excipients include diluents, binders, disintegrants, super disintegrants, solubilizers, surfactants, enzyme inhibitors, anti-adherents, anticoagulants, antifoaming agents, antioxidants, buffers chelating agents, coagulants, colorants, opaquants hydrogen bonding agents, flavorants, desensitizers, ion-exchange resins, plasticizers, preservatives, solvents, sweeteners, thickeners, and mixtures.

Typical, non-limiting examples of suitable diluents include microcrystalline cellulose, mannitol, calcium phosphate, calcium sulfate, kaolin, dry starch, powdered sugar and the like.

Typical, non-limiting examples of suitable binders include povidone, starch, stearic acid, gums, hydroxypropylmethyl cellulose and the like.

Typical, non-limiting examples of suitable disintegrants include croscarmellose sodium, crospovidone, sodium starch glycolate and the like.

Typical, non-limiting examples of suitable surfactants include polyoxyethylene glycerol esters of fatty acids, such as Tagats; polyoxylated castor oil, ethylene glycol esters, such as glycol stearate and distearate; propylene glycol esters, such as propylene glycol myristate; glyceryl esters of fatty acids, such as glyceryl stearates and monostearates; sorbitan esters, such as spans and tweens; polyglyceryl esters, such as polyglyceryl 4-oleate; fatty alcohol ethoxylates, such as Brij type emulsifiers; ethoxylated propoxylated block copolymers, such as poloxamers; polyethylene glycol esters of fatty acids, such as Labrafils, Labrafacs, and Labrasols; cremophores; glycerol monocaprylate/caprate, such as Campmul CM 10; Gelucire, Capryol, Captex, Acconon, transcutol, triacetin, TPGS (d-alpha tocopheryl polyethylene glycol succinate) and the like.

Typical, non-limiting examples of suitable lubricants include magnesium stearate, zinc stearate, calcium stearate, stearic acid, sodium stearyl fumarate, hydrogenated vegetable oil, glyceryl behenate and the like.

Typical, non-limiting examples of suitable glidants include colloidal silicon dioxide, talc or cornstarch and the like.

The modified release dosage form according to this invention comprises one or more therapeutically active ingredient. Typical, non-limiting examples of therapeutically active ingredients include Sedatives, Hypnotics, Anti-inflammatory agents, Antibiotics, Antidiabetics, Antihypertensives, Anti-Osteoporosis Agents, Antithrombotic Agents, Antivirals, Antifungals, Anticholinergic Agents, Anxiolytic Agents, Adrenergics, Antipsychotics, Anti-Parkinsonism Agents, Anticonvulsants, CNS Stimulants, Antianginal Agents, Antiarrhythmics, Antihyperlipidemic Drugs, Diuretics, Antiasthmatics, Anticoagulants, Antianemia Agents, Vitamins, Hormones, Antihistaminics, Anticancer Agents, Antiallergics, Antiarthritis Agents, Antialzheimers' Agents, Vasopressin Antagonists, Anticonvulsants, Steroids, Anesthetics, Thrombolytics, Antacids, Proton Pump Inhibitors, Protease Inhibitors, Platelet Aggregation Inhibitors, Mucolytics, Antimalarials, Antiemetics, Laxatives, Expectorants, Enzymes, Contraceptives, Bronchodilators, Antitussives, Antimigraine Agents, Anthelmintics, Anorexiants, and Antiepileptics.

In some embodiments, the therapeutically active ingredient include one or more of Non-limiting examples of suitable therapeutically active pharmaceutical ingredient may also comprise one or more of Lamotrigine, Amlodipine, Diazepam, Paracetamol, Aspirin, Ciprofloxacin, Dicyclomine, Celecoxib, Alendronate, Diacerein, Acyclovir, Fluconazole, Epinephrine, Divalproex, Methylphenidate, Flecamide, Metoprolol, Fenofibrate, Hydrochlorothiazide, Montelukast, Bicalutamide, Donepezil, Tolvaptan, Saquinavir, Bromhexine, Promethazine, Bisacodyl, Pancreatin, Ethinyl Estradiol, Salbutamol, Diphenhydramine, Sumatriptan, Diclofenac, Metronidazole, Orlistat, Ibuprofen, Indomethacin, Ketorolac, Tramadolol, Oxcarbazepine, Pioglitazone, Rosiglitazone, Miglitol, Vildagliptin, Sitagliptin, Repaglinide, Voglibose, Alprazolam, Chlorpromazine, Cimetidine, Pseudoephedrine, Naproxen, Piroxicam, Atenolol, Benazepril, Captopril, Lisinopril, Fosinopril, Enalapril, Furosemide, Indapamide, Atenolol, Felodipine, Cartenolol, Carvedilol, Cerivastatin, Diltiazem, Fluvastatin, Irbesartan, Candesartan, Methyldopa, Reserpine, Bupropion, Fluoxetine, Paroxetine, Escitalopram, Sertraline, Amitryptiline, Imipramine, Fexofenadine, Clopidogrel, Entacapone, Levodopa, Carbidopa, Levetiracetam, Venlafaxine, Duloxetine, Lisinopril, Losartan, Lovastatin, Niacin, Pravastatin, Ramipril, Simvastatin, Atorvastatin, Valsartan, Telmisartan, Sildenafil, Tadalafil, Vardenafil, Esomeprazole, Famotidine, Omeprazole, Pantoprazole, Rabeprazole, Ranitidine, Simethicone, Artesunate, Amodiaquine, Benazepril, Misoprostol, Metformin, Glipizide, Diltiazem HCl, Verapamil HCl, Labetalol HCl, Theophylline, Diclofenac Sodium, Aceclofenac, Naproxen sodium, Bupropion HCl, Metformin HCl, Duloxetine, Metoprolol tartarate & succinate, Fexofinadine HCL, Pseudoephedrine HCL, Zolpidem tartarate, Tramadol HCl, Oxybutynin chloride, Alfuzosin, including pharmaceutically acceptable salts, hydrates, solvates, esters, prodrugs thereof.

The dosage form according to this invention can be prepared in various forms including, without any limitation, a tablet, minitablets, caplet or pill.

The release of therapeutically active pharmaceutical ingredient from the dosage form according to this invention is a function of extent of polymeric coating on the inner radial surface. When the dosage form comes in contact with gastrointestinal fluid or any liquid, the fluid or liquid enters the core through the uncoated inner radial surface. The swellable polymer in the core swells upon ingestion of fluid or liquid. Upon swelling, the pressure is built up in the core of the dosage form, which induces breaking or disruption of the partial coating of the inner radial surface. This results in controlled release of the therapeutically active ingredient from the inner radial surface of the dosage form. The penetrating fluid causes release of the therapeutically active pharmaceutical ingredient in a gradual and prolonged manner by the process of dissolution of the therapeutically active pharmaceutical ingredient in the penetrating fluid and diffusion of the dissolved therapeutically active pharmaceutical ingredient back out of the core through the partial coating on inner radial surface of the dosage form. Thus the technology according to this invention achieves consistent release pattern through the designated inner radial surface of the dosage form with desirable dissolution profile. The dosage form developed helps to control initial burst release of therapeutically active pharmaceutical ingredient in gastric media and also provides further controlled release along the gastrointestinal tract.

In some embodiment of this invention, the dosage form has an in vitro dissolution profile in which at least 10% of the Lamotrigine active ingredient is dissolved in 10 hours in Phosphate buffer pH 6.8.

The pharmaceutical composition may be prepared by processes known to one of ordinary skill in the art and include but are not limited to dry granulation, wet granulation and direct compression. In one such typical, non-limiting process, one or more therapeutically active pharmaceutical ingredients are mixed with one or more pharmaceutically acceptable excipients, the blend is granulated, followed by drying and blending with lubricant, compressing the blend into a core in such a way that the resulting core has a hole extending through the core. This is then coated with one or more rate controlling polymers to form a dosage form that has a hole extending though the dosage form.

It will be readily apparent to one skilled in the art that varying substitutions and modifications may be made to the invention disclosed herein without departing from the scope and spirit of the invention. For example, those skilled in the art will recognize that the invention may be practiced using a variety of different compounds within the described generic descriptions.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

TABLE 1
Lamotrigine Extended Release Tablets

| Sr. | | Quantity |
|---|---|---|
| No. | Ingredient | (% w/w) |
| | Core formula | |
| | Intragranular | |
| 1 | Lamotrigine USP | 13.86 |
| 2 | Methocel K100 Premium CR | 16.36 |
| 3 | Methocel E4M Premium CR | 23.30 |
| 4 | Lactose monohydrate | 42.92 |
| | Binding solvent | |
| 5 | Purified water | q.s |
| | Extragranular | |
| 6 | Magnesium stearate | 0.48 |
| | Coating formula | |
| | Enteric coating | |
| 1 | Methacrylic copolymer type C | 1.79 |
| 2 | Triethyl citrate | 0.18 |
| 3 | Talc | 0.89 |
| 4 | Polysorbate 80 | 0.003 |
| 5 | Iron oxide Yellow | 0.033 |
| 6 | Purified water | q.s |

### Procedure:

In a typical example, Lamotrigine Extended Release Tablets (25 mg, 50 mg, 100 mg and 200 mg) were prepared using the formula given in Table 1. Typically, Lamotrigine, Methocel (both grades) and lactose sifted and mixed in a rapid mixing granulator using purified water. The granules were dried using suitable fluidized bed dryer followed by milling using a suitable mulitimill. The granules were sized using 20# mesh ASTM (840 micron size mesh) and lubricated in double cone blender. The blend was compressed to form core tablets having a hole extending from top to bottom of the tablet. The tablet is coated using polymers like Opadry and enteric polymers like Methacrylic copolymer type C (Eudragit L 30 D55) using suitable additives like Triethyl citrate, Polysorbate 80, Iron oxide Yellow, Talc, etc. The Lamotrigine extended release tablets obtained had a donut shape as shown in FIG. 1(a) with partially coated inner radial surface.

Table 2 compares dissolution profiles of coated tablets using USP Type II (paddle) dissolution apparatus. Typically, the dissolution tests were conducted in 900 ml of 0.1 N HCl at 50 rpm at a temperature of 37° C.±0.5° C. Tablet A is a coated tablet dosage form without any hole in the dosage form. Tablet B is a coated tablet dosage form with a hole extending through the tablet dosage form (2.5 mm central hole). Tablets A and B were coated with polymeric coat that was 3% w/w of the core weight and were obtained using a 9 mm diameter circular punch.

TABLE 2
Comparative dissolution profiles using USP Type II (Paddle) dissolution
apparatus

| | Tablet A | Tablet B |
|---|---|---|
| | (Tablet without any hole) | (Tablet with 2.5 mm central hole) |
| Time | % Release | % Release |
| 0 hr | 0 | 0 |
| 1 hr | 0 | 2 |
| 3 hr | 0 | 5 |
| 5 hr | 1 | 6 |
| 8 hr | 3 | 12 |
| 10 hr | 4 | 15 |
| 12 hr | 6 | 17 |
| 15 hr | 9 | 21 |
| 18 hr | 11 | 23 |

Table 3 provides comparative dissolution data for Tablets A and B using USP Type III (Reciprocating Cylinders) Dissolution Apparatus. Typically, the dissolution tests were conducted in 250 ml of 0.1N HCL followed by Acetate buffer (pH 4.5), Acetate buffer (pH 5.5) and Phosphate buffer (pH6.8) at 15 DPM at a temperature of 37° C.±0.5° C.

TABLE 3
Comparative dissolution profiles using USP Type III (Reciprocating
cylinders) dissolution apparatus

| | Tablet A | Tablet B |
|---|---|---|
| | (Tablet without any | (Tablet with 2.5 mm |
| | hole) | central hole) |
| Time (Dissolution Media) | % Release | % Release |
| 2 hr (0.1N HCl) | 0 | 4 |
| 2 hr (Acetate, pH 4.5) | 0 | 7 |
| 2 hr (Acetate, pH 5.5) | 3 | 12 |
| 2 hr (Phosphate, pH 6.8) | 15 | 31 |
| 2 hr (Phosphate, pH 6.8) | 35 | 49 |
| 5 hr (Phosphate, pH 6.8) | 57 | 96 |

As can be seen from the dissolution data in Tables 2-3, the tablets according to the present invention (having hole extending through the dosage form) provide a substantially modified release of Lamotrigine active ingredient.

### Example 2

TABLE 4
Alfuzosin hydrochloride Extended Release Tablets 10 mg

| Sr. | | Quantity |
|---|---|---|
| No. | Ingredient | (% w/w) |
| | Core formula | |
| 1 | Alfuzosin Hydrochloride | 5.0 |
| 2 | HPMC | 45.0 |
| 3 | Hydroxy propyl cellulose | 27.5 |
| 4 | Mannitol | 21.25 |
| 5 | Colloidal silicon oxide | 0.5 |
| 6 | Hydrogenated Castor oil | 0.25 |
| 7 | Magnesium stearate | 0.1 |
| | Extragranular | |
| 8 | Hydrogenated Castor oil | 0.25 |
| 9 | Magnesium stearate | 0.15 |
| | Coating formula | |
| 1 | Methacrylic copolymer type C | 75.0 |
| 2 | Triethyl citrate | 10.0 |
| 3 | Talc | 14.7 |
| 4 | Yellow Iron oxide | 0.3 |
| 5 | Purified water | q.s |

### Procedure

In a typical example, Alfuzosin Hydrochloride extended release tablets (10 mg) were prepared using formula given in Table 4. Typically Alfuzosin Hydrochloride, HPMC, Hydroxy propyl cellulose, Mannitol and Colloidal silicon oxide were sifted and mixed together. The blend was lubricated with Hydrogenated Castor oil and magnesium stearate in blender. This mixture was compacted and granules were formed. The granules were further lubricated with Hydrogenated Castor oil and magnesium stearate and compressed to form donut shaped tablet having a hole extending from top to bottom. The tablets were coated using Methacrylic copolymer type C (Eudragit L 30D 55). The Alfuzosin hydrochloride extended release tablets obtained had a donut shape as shown in FIG. 1(a) with partially coated inner radial surface.

Tablet 5 compares the dissolution profiles of coated tablets using USP Type II (paddle) dissolution apparatus. Typically the dissolution tests were conducted in 900 ml of 0.1 N Hydrochloric acid as a dissolution media with rotation speed of 100 rpm at a temperature of 37° C.±0.5° C. Tablet A1 is a coated tablet dosage form without any hole in the dosage form. Tablet B1 is a coated tablet dosage form with a hole extending through the tablet dosage form (2.5 mm central hole). Tablets A1 and B1 were coated with polymeric coat that was 3% w/w of the core weight and were obtained using a 9 mm diameter circular punch.

TABLE 5
Comparative dissolution profiles using Type II (Paddle) dissolution apparatus

| | Tablet A1 | Tablet B1 |
|---|---|---|
| | (Tablet without any hole) | (Tablet with 2.5 mm central hole) |
| Time | % Release | % Release |
| 1 hr | 20 | 1 |
| 2 hr | 32 | 3 |
| 5 hr | 64 | 8 |
| 10 hr | 87 | 19 |
| 12 hr | 94 | 27 |
| 15 hr | 98 | 51 |
| 20 hr | 100 | 84 |
| 24 hr | 101 | 92 |

As can be seen from the dissolution data in Table 5, the tablets according to the present invention (having hole extending through the dosage form) provide a substantially modified release of therapeutically active ingredient i.e. Alfuzosin Hydrochloride.

### Example 3

TABLE 6
Doxazosin Mesylate Extended Release Tablets 150 mg

| Sr. | | Quantity |
|---|---|---|
| No. | Ingredient | (% w/w) |
| | Core formula | |
| 1 | Doxazosin mesylate | 0.8 |
| 2 | Polyox WSR coagulant | 33.0 |
| 3 | Xanthan gum | 11.0 |
| 4 | Lactose monohydrate | 49.2 |
| 5 | Povidone | 5.0 |
| 6 | Isopropyl alcohol | q.s |
| | Magnesium stearate | 1.0 |
| | Coating formula | |
| 1 | Cellulose acetate | 95.0 |
| 2 | Polyethylene glycol 3350 | 5.0 |
| 3 | Acetone | q.s |
| 4 | Isopropyl alcohol | q.s |

### Procedure

In a typical example, Doxazosin mesylate extended release tablets (150 mg) were prepared using the formula given in Table 6. Typically, Doxazosin mesylate, Polyox WSR coagulant, Lactose monohydrate and Xanthan gum were sifted and mixed together. This mixture was granulated using Povidone. Granules were dried and lubricated with suitable lubricant and compressed to form triangular shaped tablet having a hole extending from top to bottom. The tablets were further coated using cellulose acetate coating solution. The Doxazosin mesylate extended release tablets obtained had a triangular shape as shown in FIG. 3(a) with partially coated inner radial surface.

Table 7 compares the dissolution profiles of coated tablets using USP Type II dissolution apparatus. Typically the dissolution tests were conducted in 900 ml of 0.01 N Hydrochloric acid as a dissolution media with rotation speed of 75 rpm at a temperature of 37° C.±0.5° C.

Tablet A2 is a coated tablet dosage form having a triangular shape without any hole in the dosage form. Tablet B2 is a coated tablet dosage form having a triangular shape with a hole extending through the tablet dosage form (2.5 mm central hole). Tablets A2 and B2 were coated with polymeric coat (3% w/w of the core weight) and were obtained using a 8.2×4.5 mm triangular punch.

TABLE 7
Comparative dissolution profiles using Type II dissolution apparatus

| | Tablet A2 | Tablet B2 |
|---|---|---|
| | (Tablet without any | (Tablet with 2.5 mm central |
| | hole) | hole) |
| Time | % Release | % Release |
| 2 hr | 18 | 7 |
| 4 hr | 38 | 18 |
| 8 hr | 69 | 18 |
| 12 hr | 91 | 25 |
| 15 hr | 99 | 51 |
| 20 hr | 101 | 72 |
| 24 hr | 102 | 95 |

As can be seen from the dissolution data in Table 7, the tablets according to the present invention (having hole extending through the dosage form) provide a substantially modified release of therapeutically active ingredient i.e. Doxazosin mesylate.

### Example 4

TABLE 8
Bupropion Hydrochloride Extended Release Tablets 150 mg

| Sr. | | Quantity |
|---|---|---|
| No. | Ingredient | (% w/w) |
| | Core formula | |
| 1 | Bupropion Hydrochloride | 50.0 |
| 2 | HPMC | 30.0 |
| 3 | Povidone | 16.6 |
| 4 | Purified water | q.s |
| 5 | Glyceryl behenate | 3.33 |
| | Coating formula | |
| 1 | Ethyl cellulose 7 cps | 90.0 |
| 2 | Dibutyl sebacate | 10.0 |
| 3 | Dichloro methane | q.s |
| 4 | Isopropyl alcohol | q.s |

### Procedure

In a typical example, Bupropion Hydrochloride extended release tablet (150 mg) were prepared using the formula given in table 8. Typically Bupropion Hydrochloride, HPMC and Povidone were mixed together and granulated using purified water. Granules were dried and lubricated with suitable lubricant and compressed to form octagonal shaped tablets with opening extending from top to bottom. The tablets were further coated using Ethyl cellulose solution. The Bupropion Hydrochloride extended release tablet obtained had an octagonal shape as shown in FIG. 2(a) with partially coated inner radial surface.

Table 9 compares dissolution profiles of coated tablets using USP Type II dissolution apparatus. Typically the dissolution tests were conducted in 900 ml of 0.1 N Hydrochloric acid as a dissolution media with rotation speed of 50 rpm at a temperature of 37° C.±0.5° C.

Tablet A3 is a coated tablet dosage form having an octagonal shape without any hole in the dosage form. Tablet B3 is a coated tablet dosage form having an octagonal shape with a hole extending through the tablet dosage form (3 mm central hole). Tablets A3 and B3 were coated with polymeric coat (3% w/w of the core weight) and were obtained using a 13×6 mm octagonal punch.

TABLE 9
Comparative dissolution profiles using Type II dissolution apparatus

| | Tablet A3 | Tablet B3 |
|---|---|---|
| | (Tablet without any hole) | (Tablet with 3 mm center hole) |
| Time | % Release | % Release |
| 1 hr | 51 | 25 |
| 2 hr | 86 | 45 |
| 4 hr | 98 | 67 |
| 6 hr | 101 | 85 |
| 8 hr | 102 | 98 |

As can be seen from the dissolution data in Table 9, the tablets according to the present invention (having hole extending through the dosage form) provide a substantially modified release of therapeutically active ingredient i.e. Bupropion hydrochloride.

1. A modified release dosage form comprising a core coated with a polymeric coat, said polymeric coat comprising one or more rate controlling polymers, said dosage form having a hole extending through the dosage form resulting in an inner radial surface and an outer radial surface, said core comprising at least one therapeutically active ingredient, characterized in that the inner radial surface is partially coated with said polymeric coat.

2. The modified release dosage form according to claim 1, wherein ratio of outer radial surface area to the inner radial surface area is between about 20 to about 1.

3. The modified release dosage form according to claim 1, further comprising at least one pharmaceutically acceptable excipient.

4. The modified release dosage form according to claim 1, wherein the rate controlling polymer is a hydrophilic polymer, a hydrophobic polymer or a mixture thereof.

5. The modified release dosage form according to claim 4, wherein the rate controlling polymer is at least one selected from a group of rate controlling polymers comprising polyvinyl acetate, cellulose acetate, cellulose acetate butyrate, cellulose acetate propionate, ethyl cellulose, a fatty acid, a fatty acid ester, an alkyl alcohol, a wax, shellac, rosin, zein (prolamine from corn), povidone, kollidon SR, poly(meth)acrylate, microcrystalline cellulose or poly(ethylene oxide), polyuronic acid salts, cellulose ethers, xanthan gum, tragacanth gum, gum karaya, guar gum, acacia, gellan gum locust bean gum, alkali metal salts of alginic acid or pectic acid, sodium alginate, potassium alginate, ammonium alginate, hydroxypropyl cellulose, hydroxy ethyl cellulose, hydroxypropyl methyl cellulose, carboxyvinyl polymers, polymerized gelatin, shellac, methacrylic acid copolymer, cellulose butyrate phthalate, cellulose hydrogen phthalate, cellulose propionate phthalate, polyvinyl acetate phthalate (PVAP), cellulose acetate phthalate (CAP), cellulose acetate trimellitate (CAT), hydroxypropyl methylcellulose phthalate, hydroxypropyl methylcellulose acetate, dioxypropyl methylcellulose succinate, carboxymethyl ethyl cellulose (CM EC), hydroxypropyl methylcellulose acetate succinate (HPMCAS), and acrylic acid polymers and copolymers like methyl acrylate, ethyl acrylate, methyl methacrylate, ethyl methacrylate, copolymers of acrylic and methacrylic acid esters, methacrylic acid copolymer type C and methacrylic acid copolymer type A.

6. The modified release dosage form according to claim 1, wherein the therapeutically active ingredient is at least one selected from sedatives, hypnotics, antiinflammatory agents, Antibiotics, Antidiabetics, Antihypertensives, Anti-Osteoporosis Agents, Antithrombotic Agents, Antivirals, Antifungals, Anticholinergic Agents, Anxiolytic Agents, Adrenergics, Antipsychotics, Anti-Parkinsonism Agents, Anticonvulsants, CNS Stimulants, Antianginal Agents, Antiarrhythmics, Antihyperlipidemic Drugs, Diuretics, Antiasthmatics, Anticoagulants, Antianemia Agents, Vitamins, Hormones, Antihistaminics, Anticancer Agents, Antiallergics, Antiarthritis Agents, Antialzheimers' Agents, Vasopressin Antagonists, Anticonvulsants, Steroids, Anesthetics, Thrombolytics, Antacids, Proton Pump Inhibitors, Protease Inhibitors, Platelet Aggregation Inhibitors, Mucolytics, Antimalarials, Antiemetics, Laxatives, Expectorants, Enzymes, Contraceptives, Bronchodilators, Antitussives, Antimigraine Agents, Anthelmintics, Anorexiants, and Antiepileptics.

7. The modified release dosage form according to claim 1, wherein one or more therapeutically active ingredient are selected from the group consisting of Lamotrigine, Ciprofloxacin, Divalproex, Doxazosin, Methylphenidate, Metoprolol, Bisacodyl, Diclofenac, Metronidazole, Pioglitazone, Alprazolam, Naproxen, Felodipine, Carvedilol, Diltiazem, Fluvastatin, Bupropion, Fluoxetine, Paroxetine, Fexofenadine, Entacapone, Levodopa, Carbidopa, Levetiracetam, Venlafaxine, Duloxetine, Lovastatin, Simvastatin, Esomeprazole, Pantoprazole, Rabeprazole, Misoprostol, Metformin, Glipizide, Verapamil HCl, Theophylline, Diclofenac Sodium, Naproxen sodium, Bupropion HCl, Metformin HCl, Duloxetine, Metoprolol tartarate & succinate, Fexofenadine HCL, Pseudoephedrine HCL, Zolpidem tartarate, Tramadol HCl, Oxybutynin chloride, Alfuzosin, including pharmaceutically acceptable salts, hydrates, solvates, esters, prodrugs thereof.

8. The modified release dosage form according to claim 7, wherein the therapeutically active ingredient is selected from the group consisting of Lamotrigine, Bupropion, Doxazosin, Alfuzosin and combination thereof.

9. The modified release pharmaceutical dosage form according to claim 3, wherein the pharmaceutically acceptable excipient is at least one selected from the group consisting of polymers, diluents, binders, disintegrants, surfactants, solubilizers, buffers, plasticizers, glidants, and lubricants.

10. The modified release pharmaceutical dosage form according to claim 1, wherein the dosage form is tablet, minitablets, caplet or pill.

11. The modified release dosage form according to claim 1, wherein shape of the dosage form comprise one or more of circular, oval, donut, rectangular, triangular, cylindrical, pentagonal, hexagonal, heptagonal, octagonal, trapezoid, pillowed shaped or a centrally notched round tablet.

12. A modified release dosage form comprising a core coated with a polymeric coat, said polymeric coat comprising one or more rate controlling polymers, said dosage form having a hole extending through the dosage form resulting in an inner radial surface and an outer radial surface, said core comprising Lamotrigine active ingredient, characterized in that the inner radial surface is partially coated with said polymeric coat.

13. The modified release dosage form according to claim 12, wherein ratio of outer radial surface area to the inner radial surface area is between about 20 to about 1.

14. The modified release dosage form according to claim 12, further comprising at least one pharmaceutically acceptable excipient.

15. The modified release dosage form according to claim 12, wherein the rate controlling polymer is a hydrophilic polymer, a hydrophobic polymer or a mixture thereof.

16. The modified release dosage form according to claim 15, wherein the rate controlling polymer is at least one selected from a group of rate controlling polymers comprising polyvinyl acetate, cellulose acetate, cellulose acetate butyrate, cellulose acetate propionate, ethyl cellulose, a fatty acid, a fatty acid ester, an alkyl alcohol, a wax, shellac, rosin, zein (prolamine from corn), povidone, kollidon SR, poly(meth)acrylate, microcrystalline cellulose or poly(ethylene oxide), polyuronic acid salts, cellulose ethers, xanthan gum, tragacanth gum, gum karaya, guar gum, acacia, gellan gum locust bean gum, alkali metal salts of alginic acid or pectic acid, sodium alginate, potassium alginate, ammonium alginate, hydroxypropyl cellulose, hydroxy ethyl cellulose, hydroxypropyl methyl cellulose, carboxyvinyl polymers, polymerized gelatin, shellac, methacrylic acid copolymer, cellulose butyrate phthalate, cellulose hydrogen phthalate, cellulose propionate phthalate, polyvinyl acetate phthalate (PVAP), cellulose acetate phthalate (CAP), cellulose acetate trimellitate (CAT), hydroxypropyl methylcellulose phthalate, hydroxypropyl methylcellulose acetate, dioxypropyl methylcellulose succinate, carboxymethyl ethyl cellulose (CM EC), hydroxypropyl methylcellulose acetate succinate (HPMCAS), and acrylic acid polymers and copolymers like methyl acrylate, ethyl acrylate, methyl methacrylate, ethyl methacrylate, copolymers of acrylic and methacrylic acid esters, methacrylic acid copolymer type C and methacrylic acid copolymer type A.

17. The modified release pharmaceutical dosage form according to claim 14, wherein the pharmaceutically acceptable excipient is at least one selected from the group consisting of polymers, diluents, binders, disintegrants, surfactants, solubilizers, buffers, plasticizers, glidants, and lubricants.

18. The modified release dosage form according to claim 12, wherein the dosage form has an in vitro dissolution profile in which at least about 10% of the Lamotrigine active ingredient is dissolved in 10 hours in Phosphate buffer pH 6.8.

19. The modified release pharmaceutical dosage form according to claim 12, wherein the dosage form is tablet, minitablets, caplet or pill.

20. The modified release dosage form according to claim 12, wherein shape of the dosage form comprise one or more of circular, oval, donut, rectangular, triangular, cylindrical, pentagonal, hexagonal, heptagonal, octagonal, trapezoid, pillowed shaped or a centrally notched round tablet.
